(19) United States
(12) Patent Application Publication
(10) Pub. No.: US 2015/0165438 A1
(43) Pub. Date: Jun. 18, 2015

(54) MICROFLUIDIC DEVICE AND TEMPERATURE CONTROL METHOD FOR MICROFLUIDIC DEVICE

(71) Applicant: CANON KABUSHIKI KAISHA, Tokyo (JP)

(72) Inventor: Hirotomo Taniguchi, Saitama-shi (JP)

(21) Appl. No.: 14/565,111

(22) Filed: Dec. 9, 2014

(30) Foreign Application Priority Data
Dec. 13, 2013 (JP) 2013-258047

(51) Int. Cl. B01L 7/00; C12Q 3/00

(52) U.S. Cl. CPC B01L 7/52; C12Q 3/00

(57) ABSTRACT

A microfluidic device includes a resistor. The resistor serves both as a heater configured to heat a fluid flowing inside a flow channel provided in a base and as a sensor configured to measure a temperature of the flow channel. A temperature sensor configured to measure a change in an ambient temperature is arranged in an outer side portion positioned in a longitudinal direction of the resistor. A temperature erroneously determined in a case where the value of resistance of the resistor in an expression 1 is affected by a change in the ambient temperature is corrected in accordance with a change in the ambient temperature measured by the temperature sensor, and the temperature of the region of interest is controlled.

### BACKGROUND OF THE INVENTION

1. Field of the Invention

The present invention relates to a microfluidic device and a temperature control method for the microfluidic device. The present invention relates more specifically to a microfluidic device especially having a micro flow channel and used to perform chemosynthesis, genetic testing, and the like in accordance with chemical, biochemical, physical chemical reaction, and the like, and a temperature control method for the microfluidic device.

2. Description of the Related Art

Hitherto, chemosynthesis, genetic testing, genetic research and development, and the like have been studied actively using a microfluidic device having a micro flow channel in accordance with chemical, biochemical, physical chemical reaction, and the like.

As such a microfluidic device, IEEJ Transactions on Sensors and Micromachines, Vol. 119 (1999), No. 10, pp. 448-453 and Japanese Patent Laid-Open No. 2004-33907 have disclosed a microfluidic device which has a micro flow channel, in the microfluidic device a heater and a temperature sensor being arranged in the same base as the micro flow channel so as to heat a fluid inside the micro flow channel, and which controls the output of the heater using the temperature sensor and performs control such that the temperature of the micro flow channel reaches a desired temperature.

With reference to FIG. 12, the configuration of a microfluidic device of the above-described conventional example will be described.

In FIG. 12, reference numeral 1 denotes a supporting base, reference numeral 2 denotes a base where a flow channel is formed, reference numeral 3 denotes a flow channel, and reference numeral 4 denotes a resistor that has both the function of a heater that heats a fluid inside the flow channel and a function for measuring the temperature of the flow channel.

The value of resistance of the resistor changes with temperature, so a change in temperature may be measured by a change in resistance.

Reference numeral 5 denotes an inlet for a fluid, reference numeral 6 denotes an outlet, reference numeral 7 denotes an electrode wiring line, reference numeral 8 denotes an electrode pad for electrical conduction, and reference numeral 9 denotes a heat sink, which is a cooling mechanism.

Reference numeral 10 denotes a region of interest, a temperature measurement region, where the temperature in the flow channel is controlled.

The fluid in the flow channel is heated by heat conduction based on heat as Joule heat generated by applying a voltage to the resistor 4.

A relationship between the temperature of the region of interest 10 and the value of resistance of the resistor 4 is obtained by, in advance, applying an appropriate voltage to the resistor 4, by measuring the temperature of the region of interest 10 with an infrared radiation thermometer or the like when the appropriate voltage is applied to the resistor 4, and by associating the temperature of the region of interest 10 with the value of resistance of the resistor 4.

The temperature of the region of interest 10 is controlled in accordance with the value of resistance of the resistor 4, using the relationship between the temperature of the region of interest 10 and the value of resistance of the resistor 4.

The case where a resistor serving as a heater of the above-described conventional example is used as a sensor that measures the temperature of a flow channel has a problem such as that described below.

In order to make the temperature of a region of interest uniform in the longitudinal direction of a flow channel, it is necessary to arrange a resistor that is long with respect to the region of interest. Here, in the case where a sensor that measures the temperature of the flow channel is arranged near a region of interest of the flow channel, it is difficult to do sensor wiring line layout when a plurality of flow channels are arranged in a limited space.

In addition, in the case where the resistor also serving as a heater is used as a sensor that measures the temperature of the flow channel, there has been a problem such as that described below.

As illustrated in a graph of FIG. 7, a temperature distribution exists in the longitudinal direction of the heater and the flow channel.

This temperature distribution changes with the ambient temperature of the microfluidic device. When the temperature distribution changes, even though the temperature of the region of interest does not change, the value of resistance of the entirety of the resistor changes. As a result, a controller determines that the temperature of the region of interest has changed and controls the output of the heater.

Then, as illustrated in FIG. 8, for example, when the outside temperature changes from A to B, the controller erroneously determines that the temperature of the region of interest has increased. As a result, the controller performs control such that the temperature of the region of interest is made lower than a target temperature.

In chemosynthesis and genetic testing performed in a micro flow channel, it is necessary to control temperature with high accuracy. There may be a case where slightly erroneous control of temperature affects the above-described chemosynthesis and genetic testing to a significant degree.

### SUMMARY OF THE INVENTION

The present invention provides a microfluidic device that makes it possible to measure the temperature of a fluid in a flow channel included in the microfluidic device with high accuracy and control the temperature of the fluid, and a temperature control method for the microfluidic device.

A microfluidic device according to the present invention is a microfluidic device including a controller configured to control a temperature of a region of interest, which is a temperature measurement region, in accordance with a value of resistance of a resistor in an expression 1 in which the value of resistance of the resistor is associated with a temperature of the region of interest.

The resistor serves both as a heater configured to heat a fluid flowing inside a flow channel provided in a base of the microfluidic device and as a sensor configured to measure a temperature of the flow channel.

The resistor is provided so as to extend over a region wider than the region of interest along a longitudinal direction of the flow channel and near the flow channel including the region of interest.

A temperature sensor configured to measure a change in an ambient temperature is arranged in an outer side portion positioned in a longitudinal direction of the resistor.

A temperature erroneously determined in a case where the value of resistance of the resistor in the expression 1 is affected by a change in the ambient temperature is corrected in accordance with a change in the ambient temperature measured by the temperature sensor, and the temperature of the region of interest is controlled.

A temperature control method for a microfluidic device according to the present invention includes controlling a temperature of a region of interest, which is a temperature measurement region, in accordance with a value of resistance of a resistor in an expression 1 in which the value of resistance of the resistor is associated with a temperature of the region of interest. The resistor serves both as a heater configured to heat a fluid flowing inside a flow channel provided in a base of the microfluidic device and as a sensor configured to measure a temperature of the flow channel.

The resistor is provided so as to extend over a region wider than the region of interest along a longitudinal direction of the flow channel and near the flow channel including the region of interest.

A temperature sensor configured to measure a change in an ambient temperature is arranged in an outer side portion positioned in a longitudinal direction of the resistor.

A temperature erroneously determined in a case where the value of resistance of the resistor in the expression 1 is affected by a change in the ambient temperature is corrected in accordance with a change in the ambient temperature measured by the temperature sensor, and the temperature of the region of interest is controlled.

According to the present invention, there may be realized a microfluidic device that makes it possible to measure the temperature of a fluid in a flow channel included in the microfluidic device with high accuracy and control the temperature of the fluid, and a temperature control method for the microfluidic device.

Further features of the present invention will become apparent from the following description of exemplary embodiments with reference to the attached drawings.

### BRIEF DESCRIPTION OF THE DRAWINGS

FIG. 1 is a diagram used to describe an example of the configuration of a microfluidic device according to an embodiment of the present invention.

FIG. 2 is a diagram used to describe an example of the configuration of a microfluidic device according to the embodiment of the present invention.

FIG. 3 is a diagram used to describe an example of the configuration of a microfluidic device according to the embodiment of the present invention.

FIG. 4 is a diagram used to describe an expression 2 for correction of an expression 1 in the embodiment of the present invention.

FIG. 5 is a diagram used to describe a database for correction of the expression 1 in the embodiment of the present invention.

FIG. 6 is a diagram used to describe an example of a database for correction of the expression 1 and a correction method in the embodiment of the present invention.

FIG. 7 is a diagram used to describe a temperature distribution over a heater and in the longitudinal direction of a flow channel, in the description of related art of the present invention.

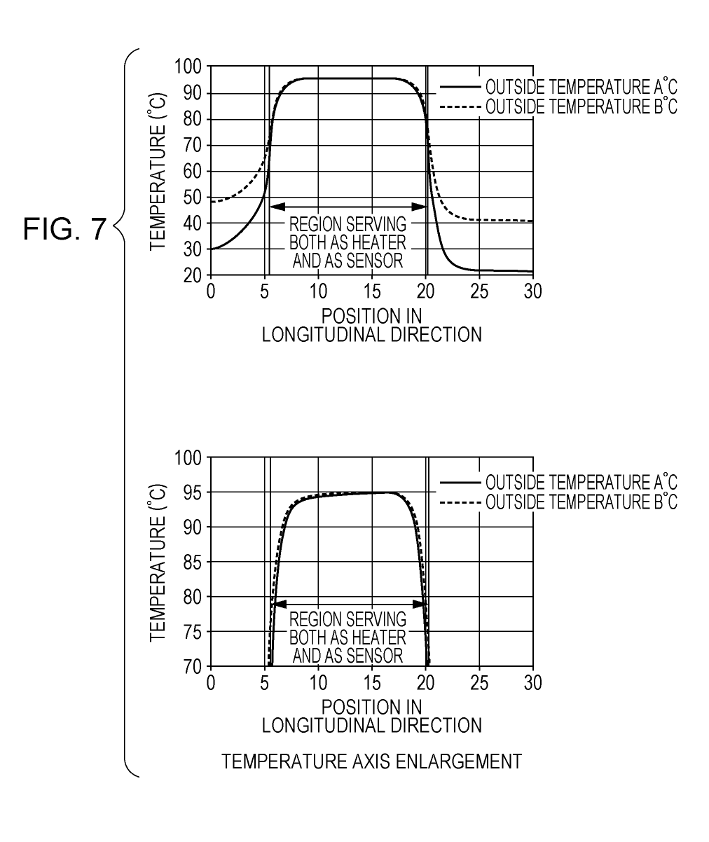

FIG. 8 is a diagram used to describe erroneous control caused by an erroneous determination as to a region of interest, in the description of related art of the present invention.

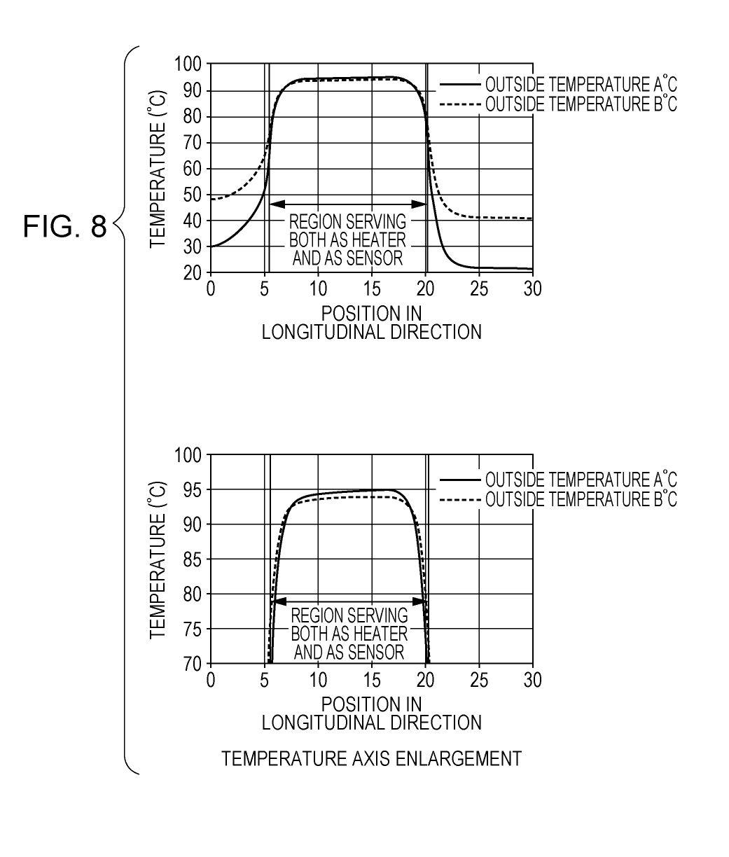

FIG. 9 is a diagram used to describe that the difference between temperature distributions is sufficiently large when the ambient temperature changes, at a position outside a cooling mechanism in the embodiment of the present invention.

FIG. 10 is a diagram illustrating a temperature distribution of a flow channel in an exemplary embodiment of the present invention.

FIG. 11 is a diagram illustrating a temperature distribution of a flow channel in a comparative example of the present invention.

FIG. 12 is a diagram used to describe a microfluidic device according to a conventional example.

FIG. 13 is a diagram used to describe an overview of correction performed in the exemplary embodiment of the present invention.

### DESCRIPTION OF THE EMBODIMENTS

Next, examples of the configuration of a microfluidic device and a temperature control method for the microfluidic device according to an embodiment of the present invention will be described.

The microfluidic device according to the embodiment includes a controller that uses an expression 1 in which the value of resistance of a resistor is associated with the temperature of a region of interest, which is a temperature measurement region. The controller corrects a temperature that is erroneously determined in the case where the value of resistance of the resistor is affected by a change in the ambient temperature when the temperature of the region of interest is controlled in accordance with the value of resistance of the resistor, and controls the temperature of the region of interest.

In that case, the resistor serves both as a heater that heats a fluid flowing inside a flow channel provided in a base of the microfluidic device and as a sensor that measures the temperature of the flow channel.

The resistor is provided so as to extend over a region wider than the region of interest along the longitudinal direction of the flow channel and near the flow channel including the region of interest.

In addition, the temperature sensor that measures a change in the ambient temperature is arranged in an outer side portion positioned in the longitudinal direction of the resistor.

Then, the controller is configured so as to correct a temperature that is erroneously determined in the case where the value of resistance of the resistor in the expression 1 is affected by a change in the ambient temperature in accordance with a change in the ambient temperature measured by the temperature sensor, and so as to control the temperature of the region of interest.

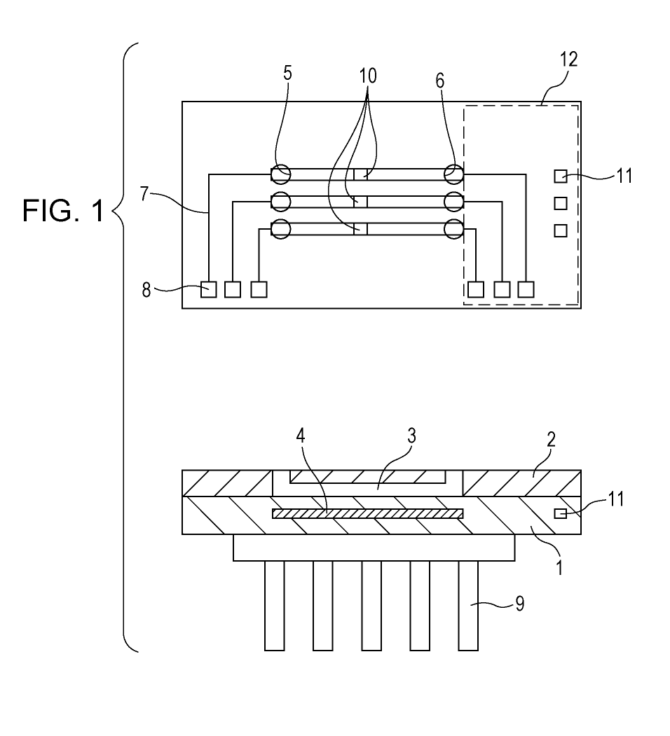

A detailed configuration is described in the following with reference to FIGS. 1 to 3.

In FIGS. 1 to 3, reference numeral 1 denotes a supporting base, reference numeral 2 denotes a base where a flow channel is formed, reference numeral 3 denotes a flow channel, reference numeral 4 denotes a resistor that serves both as a heater that heats a fluid inside the flow channel and as a sensor that measures temperature, reference numeral 5 denotes an inlet for a fluid, and reference numeral 6 denotes an outlet.

Reference numeral 7 denotes an electrode wiring line, and reference numeral 8 denotes an electrode pad for electrical conduction. Reference numeral 9 denotes a cooling mechanism (a cooling unit) for cooling a base, such as a heat sink.

Reference numeral 10 denotes a region of interest where the temperature is controlled, and reference numeral 11 is a sensor that measures a change in the ambient temperature. Reference numeral 12 denotes a region where the sensor 11 is arranged.

In each of FIGS. 1 to 3, the flow channel 3 and the resistor 4 are arranged so as to be parallel to each other in the longitudinal direction of the flow channel 3 and the resistor 4.

In a microfluidic device illustrated in FIG. 1, the sensor 11 is arranged in an outer side portion positioned in the longitudinal direction of the resistor 4.

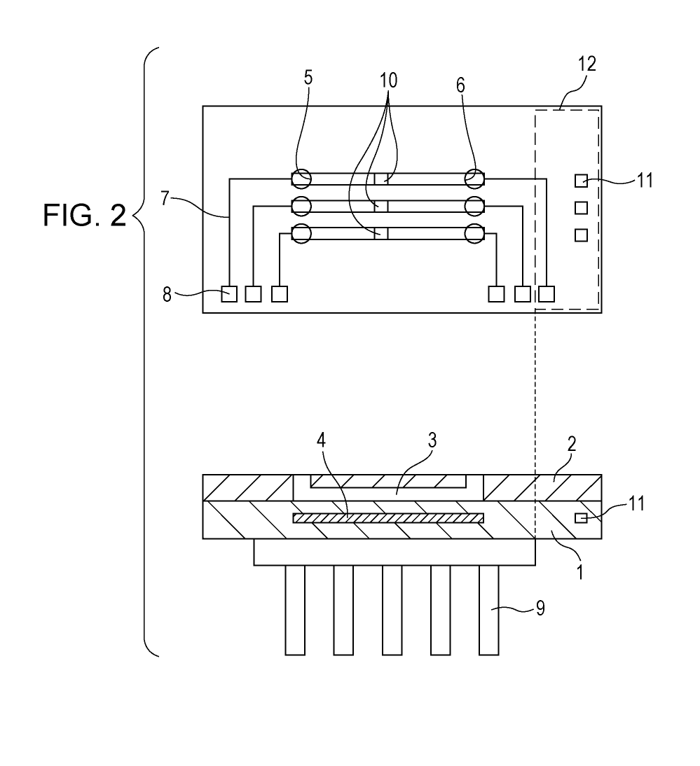

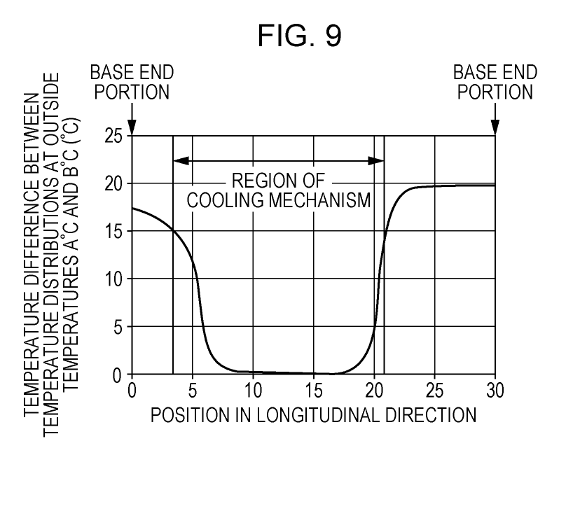

In a microfluidic device illustrated in FIG. 2, the sensor 11 is arranged outside the cooling mechanism 9 in the longitudinal direction of the resistor 4. As illustrated in FIG. 9, at a position outside the cooling mechanism 9, the difference between temperature distributions is sufficiently large when the ambient temperature changes.

This is because, in an inner side portion of the cooling mechanism 9, heat conduction is large to the cooling mechanism 9 and the temperature distribution is made uniform.

Thus, the sensitivity to a change in the ambient temperature is increased and the accuracy of measurement of a change in the ambient temperature is increased by arranging the sensor 11 outside the cooling mechanism 9.

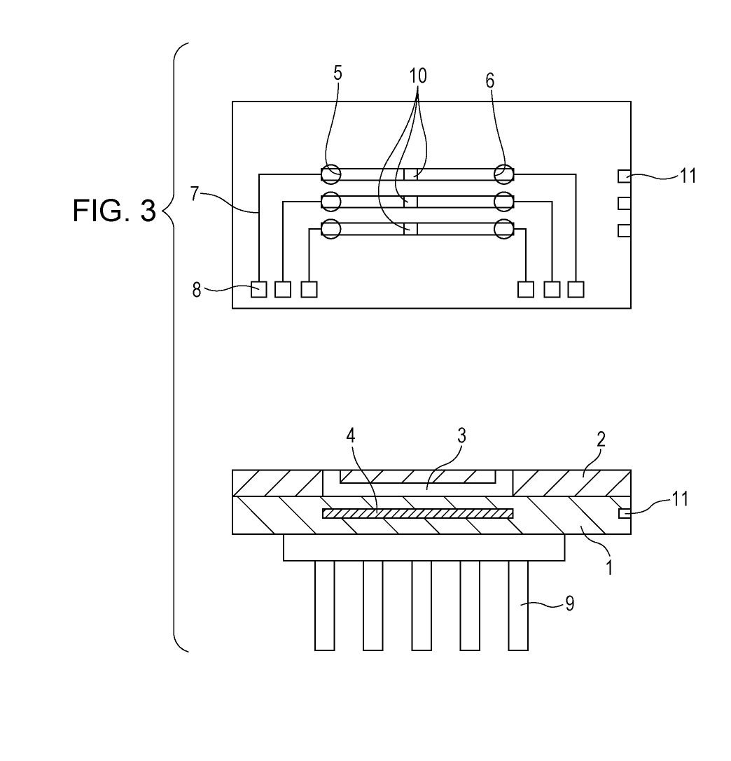

In a microfluidic device illustrated in FIG. 3, the sensor 11 is arranged in an end portion of a substrate in the longitudinal direction of the resistor 4. As illustrated in FIG. 9, the end portion of the substrate is most sensitive to a change in the ambient temperature and the accuracy of measurement of a change in the ambient temperature is increased.

As the supporting base 1, a glass material such as quartz is mainly used; however, a material other than a glass material, such as silicon and a ceramic may also be used. As the resistor 4, a metal such as platinum and ruthenium tetroxide is used. As the electrode wiring line 7, a metal such as gold and aluminum is used. As the sensor 11, platinum or the like is used.

Next, a temperature control method for the microfluidic device according to the embodiment will be described. For the microfluidic devices illustrated in FIGS. 1 to 3, a tube for an interface is connected to the inlet 5 and the outlet 6 and a fluid is injected into the inlet 5 and output from the outlet 6 by an external pump.

The fluid in a flow channel provided inside the base 2 is heated by Joule heat generated by applying a voltage to the resistor 4.

A relationship between the temperature of the region of interest 10 and the resistor 4 has been calibrated in advance, and is stored as the expression 1 described below.

R=k0+k1×T [Expression 1]

R represents the value of resistance of the resistor 4, T represents the temperature of the region of interest 10, and k₀ and k₁ are coefficients.

The expression 1 is calibrated by applying an appropriate voltage to the resistor 4, by measuring the temperature of the region of interest 10 with an infrared radiation thermometer or the like when the appropriate voltage is applied to the resistor 4, and by associating the temperature of the region of interest 10 with the value of resistance of the resistor 4.

The temperature of the flow channel is obtained in accordance with the value of resistance of the resistor 4, and a voltage to be applied to the resistor 4 is set such that the temperature of the flow channel reaches a target temperature. In accordance with a measurement value of the sensor 11 illustrated in FIGS. 1 to 3, the expression 1 regarding the temperature of the region of interest 10 and the value of resistance of the resistor 4 is corrected.

Next, a first method for correcting the expression 1 will be described in the following.

An expression 2, which is an expression regarding the temperature measured by the sensor 11 and the coefficients of the expression 1 (k₀ and k₁), has been derived from measurement or a numerical value simulation in advance and is stored in the controller.

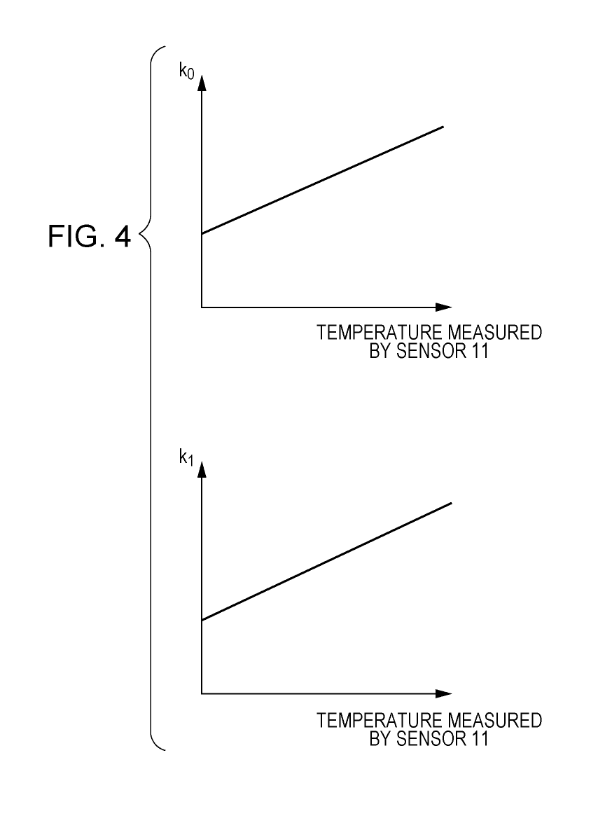

FIG. 4 includes graphs of the expression 2 regarding the temperature measured by the sensor 11 and the coefficients of the expression 1 (k₀ and k₁).

The expression 1 is corrected in accordance with the value of the ambient temperature measured by the sensor 11 and the expression 2, and the temperature of the region of interest 10 is controlled in accordance with the corrected expression 1 and the value of resistance of the resistor 4.

A second method for correcting the expression 1 will be described in the following.

The temperature measured by the sensor 11 and a plurality of coefficients of the expression 1 (k₀ and k₁) corresponding to the temperature measured by the sensor 11 have been derived from measurement or a numerical value simulation in advance, the plurality of coefficients being used to associate the value of resistance of the resistor 4 with the temperature of the region of interest 10. The plurality of coefficients are associated with the temperature measured by the sensor 11 and are stored as a database in the controller.

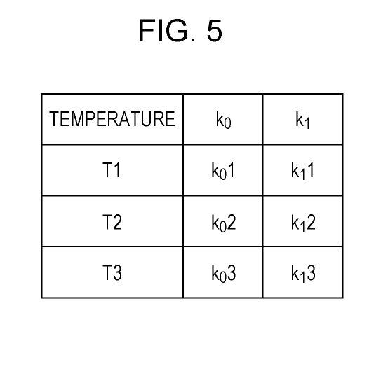

FIG. 5 illustrates an example of the database.

In the database, a value closest to the temperature measured by the sensor 11 is applied as a correction value, and the coefficients of the expression 1 (k₀ and k₁) are corrected.

The temperature of the region of interest 10 is controlled in accordance with the corrected expression 1 and the value of resistance of the resistor 4.

A third method for correcting the expression 1 will be described in the following.

The temperature measured by the sensor 11 and the coefficients of the expression 1 (k₀ and k₁) corresponding to the temperature, has been derived from measurement or a numerical value simulation in advance and are stored as a database in the controller.

The coefficients of the expression 1 at the temperature measured by the sensor 11 are corrected by interpolation of data of the database.

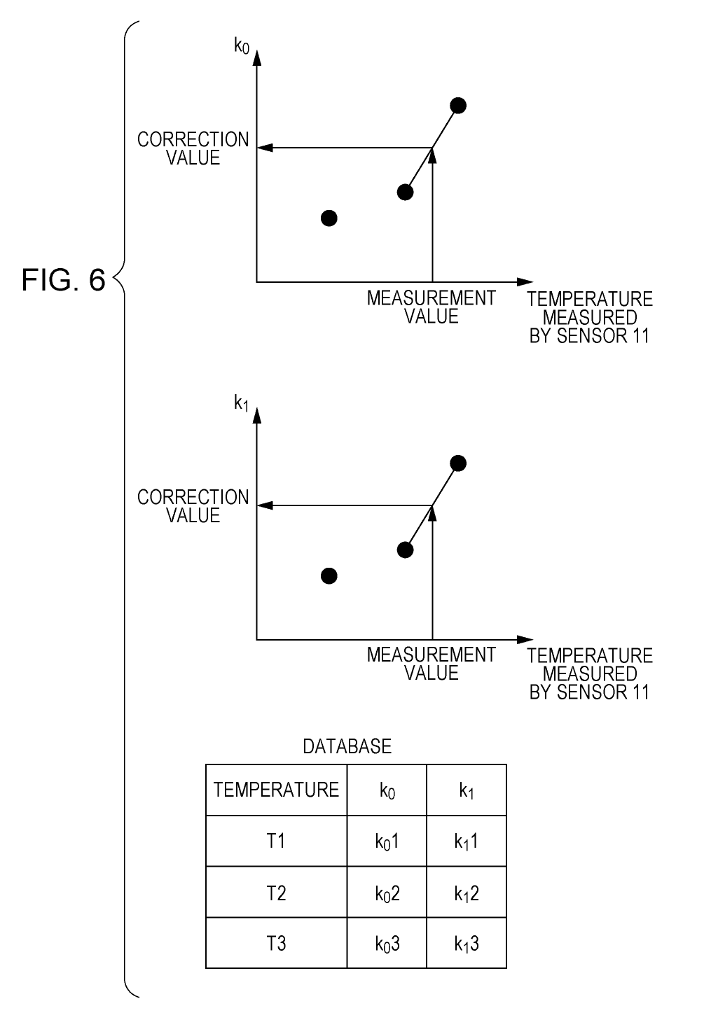

FIG. 6 is a schematic diagram of an example of the database and a correction method. The temperature of the region of interest 10 is controlled in accordance with the corrected expression 1 and the value of resistance of the resistor 4.

### Exemplary Embodiment

In the following, an exemplary embodiment of the present invention will be described.

In the exemplary embodiment, the microfluidic device having a configuration illustrated in FIG. 1 and including the supporting base 1 and the flow channel 3 was formed in the following manner.

As a material, a synthetic quartz substrate was used having a thermal conductivity of about 1.4 W/m/K at 20° C.

First, the resistor 4 serving both as a heater and as a sensor and the sensor 11 were formed on the supporting base 1. The resistor 4 was formed by forming a film of platinum having a thickness of about 100 nm by performing a sputtering method and then by forming the film so as to have a width of about 300 um by photolithography.

The sensor 11 was arranged at a position outside the heat sink 9, a cooling mechanism.

The heat sink 9 was adhered to the supporting base 1 with a double-sided adhesive tape having thermal conductivity.

Next, as an electrode wiring line of the resistor 4, a film having a thickness of about 300 nm was formed by consecutively using titanium-gold-titanium by a sputtering method, and then patterning was performed by photolithography. Next, as an insulating layer, a film of silicon oxide was formed so as to have a thickness of about 1 um by a sputtering method. Next, the electrode wiring line 7 and the electrode pad 8 were formed.

Furthermore, as an insulating layer, a film of silicon oxide was formed so as to have a thickness of about 1 um by a sputtering method. In a flow channel base 2, a flow channel was formed so as to have a width of about 200 um and a depth of about 100 um by sandblasting. The supporting base 1 and the flow channel base 2 were joined together and the microfluidic device was completed.

In the exemplary embodiment, the polymerase chain reaction (PCR) was executed, which is an amplification reaction of genes.

PCR is a method for amplifying DNA in a certain specified region.

A PCR reaction in a microfluidic device is executed by injecting a PCR solution into a flow channel of the microfluidic device and by applying a thermal cycle to the fluid in the flow channel.

The PCR solution includes components such as DNA, which is an amplification target, primers, DNA polymerases, and a buffer solution.

First, a reaction fluid is heated to about 94° C., and a double-stranded DNA is separated into single strands.

Next, the reaction fluid is rapidly cooled to about 50° C. and annealing is performed in which a primer is joined together with a single-strand DNA.

In the end, the reaction fluid is heated to 70° C., DNA polymerase is reacted, and DNA is extended.

By repeating this cycle, DNA is amplified. In general, it is said that after n cycles DNA is amplified by 2ⁿ times.

A tube for an interface was connected to the inlet 5 and the outlet 6 of the microfluidic device illustrated in FIG. 1, and a PCR reaction solution was injected into the inlet 5 and output from the outlet 6 by an external pump.

The microfluidic device used in the exemplary embodiment includes a correction device used to correct the expression 1 regarding the temperature of the region of interest 10 and the resistor 4 and a controller that controls power to be applied to the resistor 4 using the expression 1.

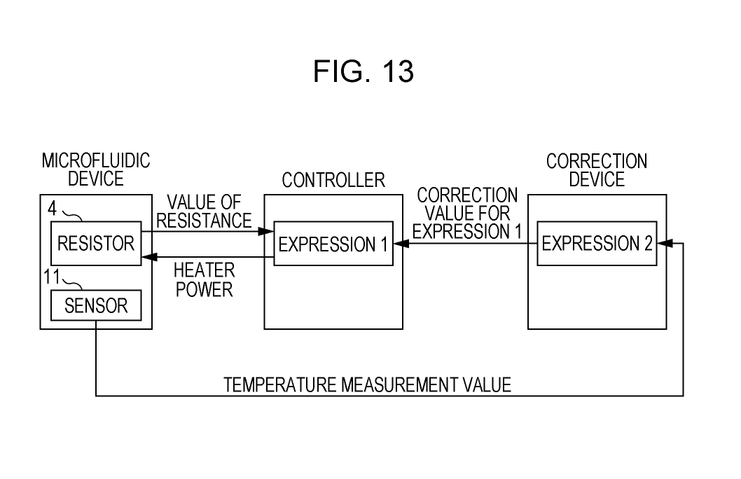

FIG. 13 illustrates an overview of correction performed in the exemplary embodiment.

The expression 1 has been calibrated in advance by applying an appropriate voltage to the resistor 4, by measuring the temperature of the region of interest 10 with an infrared radiation thermometer or the like, and by associating the temperature of the region of interest 10 with the value of resistance of the resistor 4.

The expression 2, which is an expression regarding the temperature measured by the sensor 11 and the expression 1, is stored in an arithmetic unit. The value of the temperature measured by the sensor 11 is supplied to the arithmetic unit. In the arithmetic unit, the expression 1 is corrected in accordance with the expression 2. In the controller, in accordance with the corrected expression 1 and the value of resistance of the resistor 4, power to be supplied by PID control to the resistor 4 is adjusted and the temperature of the region of interest 10 in a flow channel is controlled.

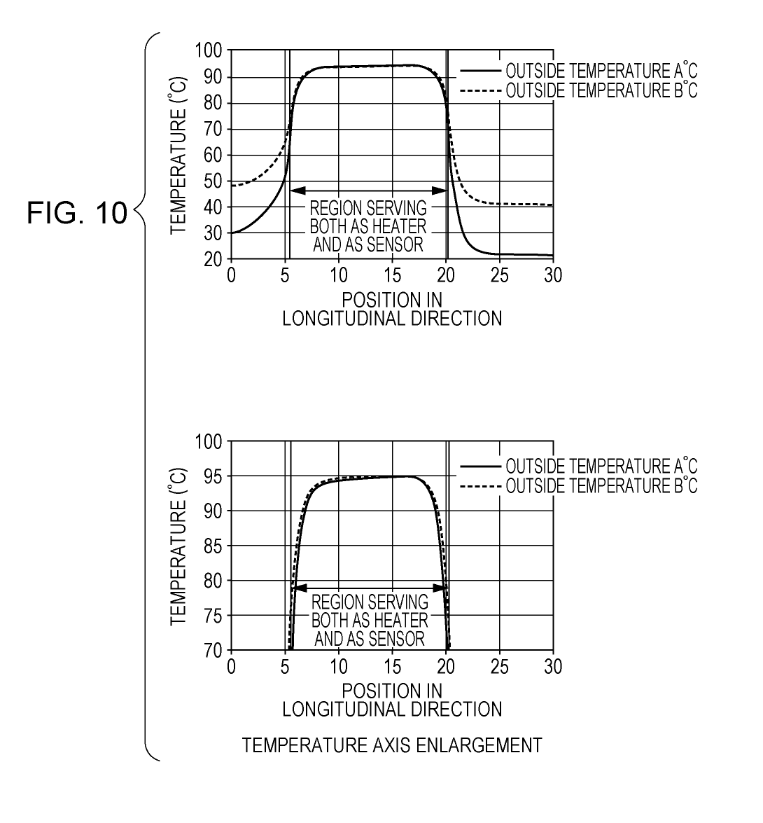

FIG. 10 illustrates a temperature distribution of a flow channel in the exemplary embodiment.

Even when the ambient temperature changed, the temperature of the region of interest 10 did not change and it was less likely that the temperature was erroneously controlled. In the PCR reaction performed using the microfluidic device of the exemplary embodiment, the PCR yield had a value nearly 100% of the value expected.

### Comparative Example

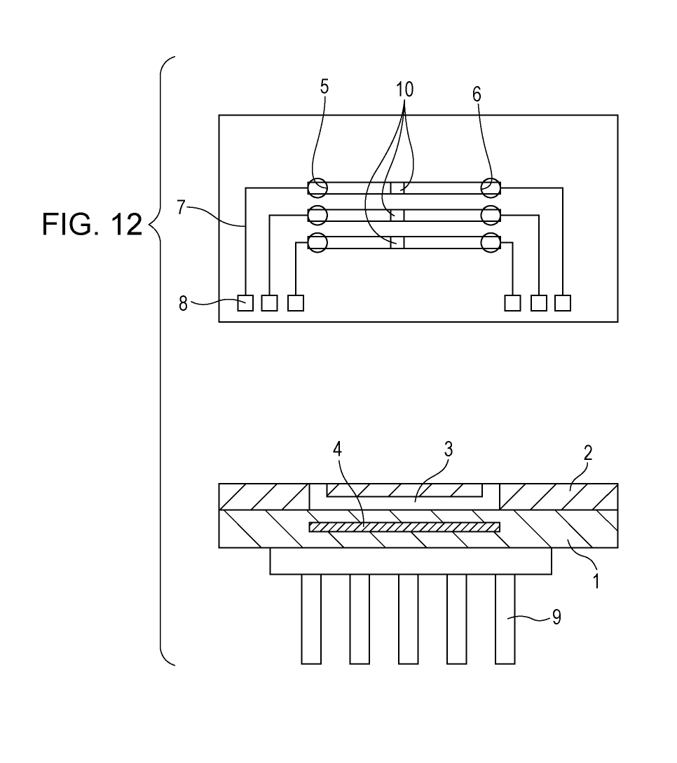

A microfluidic device used in a comparative example will be described with reference to FIG. 12.

The microfluidic device was formed in a method similar to that described in the exemplary embodiment; however, the sensor 11 illustrated in FIG. 1 is not formed.

A PCR reaction was performed in the comparative example.

The microfluidic device used in the comparative example includes a controller that controls power to be applied to the resistor 4 in accordance with the expression 1 regarding the temperature of the region of interest 10 and the resistor 4.

The expression 1 has been calibrated in advance by applying an appropriate voltage to the resistor 4, by measuring the temperature of the region of interest 10 with an infrared radiation thermometer or the like when the appropriate voltage is applied to the resistor 4, and by associating the temperature of the region of interest 10 with the value of resistance of the resistor 4.

In the controller, in accordance with the expression 1 and the value of resistance of the resistor 4, power to be supplied by PID control to the resistor 4 is adjusted and the temperature of the region of interest 10 in a flow channel is controlled.

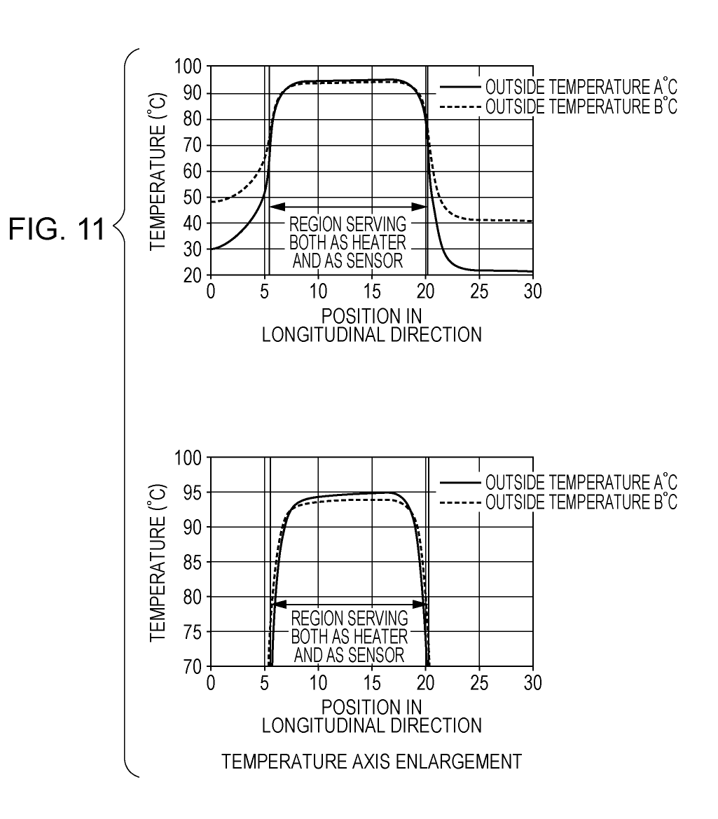

FIG. 11 illustrates a temperature distribution of a flow channel in the comparative example.

When the ambient temperature changed, the temperature was erroneously controlled and reached a temperature different from a target temperature.

In the PCR reaction performed using the microfluidic device of the comparative example, the ambient temperature changed from 20° C., which was the temperature when calibration was performed, to 40° C. and thus the PCR yield was about 50% of the value expected.

The microfluidic devices and the temperature control methods for the microfluidic device, which have been described above, may be used for a microfluidic device used to perform chemosynthesis, environment analysis, and clinical specimen analysis including a heating or cooling process.

### Other Embodiments

The present invention includes a program that causes a computer to execute the above-described temperature control method.

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-258047, filed Dec. 13, 2013 which is hereby incorporated by reference herein in its entirety.

What is claimed is:

1. A microfluidic device comprising: a controller configured to control a temperature of a region of interest, which is a temperature measurement region, in accordance with a value of resistance of a resistor in an expression 1 in which the value of resistance of the resistor is associated with a temperature of the region of interest, wherein
the resistor serves both as a heater configured to heat a fluid flowing inside a flow channel provided in a base of the microfluidic device and as a sensor configured to measure a temperature of the flow channel,
the resistor is provided so as to extend over a region wider than the region of interest along a longitudinal direction of the flow channel and near the flow channel including the region of interest,
a temperature sensor configured to measure a change in an ambient temperature is arranged in an outer side portion positioned in a longitudinal direction of the resistor, and
a temperature erroneously determined in a case where the value of resistance of the resistor in the expression 1 is affected by a change in the ambient temperature is corrected in accordance with a change in the ambient temperature measured by the temperature sensor, and the temperature of the region of interest is controlled.

2. The microfluidic device according to claim 1, wherein the base is provided with a cooling unit configured to cool the base, and
the temperature sensor is arranged outside the cooling unit in the longitudinal direction of the resistor.

3. The microfluidic device according to claim 1, wherein the temperature sensor is arranged at an end portion of the base in the longitudinal direction of the resistor.

4. The microfluidic device according to claim 1, wherein an expression 2 in which a coefficient is associated with a temperature measured by the temperature sensor is stored in the controller, the coefficient being used to associate the value of resistance of the resistor with the temperature of the region of interest, the expression 1 is corrected in accordance with the expression 2, and the temperature of the region of interest is controlled.

5. The microfluidic device according to claim 1, wherein data used to correct the erroneously determined temperature in the expression 1 is stored as a database in the controller, the data corresponding to the change in the ambient temperature measured by the temperature sensor, and
the expression 1 is corrected using the database and the temperature of the region of interest is controlled.

6. The microfluidic device according to claim 1, wherein data used to correct the erroneously determined temperature in the expression 1 is stored as a database in the controller, the data corresponding to the change in the ambient temperature measured by the temperature sensor, and
the expression 1 is corrected by interpolating data of the database and the temperature of the region of interest is controlled.

7. A temperature control method for a microfluidic device comprising:
controlling a temperature of a region of interest, which is a temperature measurement region, in accordance with a value of resistance of a resistor in an expression 1 in which the value of resistance of the resistor is associated with a temperature of the region of interest, wherein
the resistor serves both as a heater configured to heat a fluid flowing inside a flow channel provided in a base of the microfluidic device and as a sensor configured to measure a temperature of the flow channel,
the resistor is provided so as to extend over a region wider than the region of interest along a longitudinal direction of the flow channel and near the flow channel including the region of interest,
a temperature sensor configured to measure a change in an ambient temperature is arranged in an outer side portion positioned in a longitudinal direction of the resistor, and
a temperature erroneously determined in a case where the value of resistance of the resistor in the expression 1 is affected by a change in the ambient temperature is corrected in accordance with a change in the ambient temperature measured by the temperature sensor, and the temperature of the region of interest is controlled.

8. The temperature control method for a microfluidic device according to claim 7, wherein the base is provided with a cooling unit configured to cool the base, and
the temperature sensor is arranged outside the cooling unit in the longitudinal direction of the resistor.

9. The temperature control method for a microfluidic device according to claim 7, wherein the temperature sensor is arranged at an end portion of the base in the longitudinal direction of the resistor.

10. The temperature control method for a microfluidic device according to claim 7, wherein in a case where the temperature of the region of interest is controlled,
the expression 1 is corrected using an expression 2 in which a coefficient used to associate the value of resistance of the resistor with the temperature of the region of interest is associated with a temperature measured by the temperature sensor, and the temperature of the region of interest is controlled.

11. The temperature control method for a microfluidic device according to claim 7, wherein in a case where the temperature of the region of interest is controlled,
the expression 1 is corrected using a database including data with which the erroneously determined temperature in the expression 1 is corrected, the data corresponding to the change in the ambient temperature measured by the temperature sensor, and the temperature of the region of interest is controlled.

12. The temperature control method for a microfluidic device according to claim 7, wherein in a case where the temperature of the region of interest is controlled,
the expression 1 is corrected by interpolating a database including data with which the erroneously determined temperature in the expression 1 is corrected, the data corresponding to the change in the ambient temperature measured by the temperature sensor, and the temperature of the region of interest is controlled.

13. A medium in which a program that causes a computer to execute the method according to claim 7 is stored.
